(19) United States
(12) Patent Application Publication
(10) Pub. No.: US 2026/0363659 A1
(43) Pub. Date: Nov. 19, 2026

(54) FLEXOR TENDON IMAGING APPARATUS

(71) Applicant: The Regents of the University of California, Oakland, CA (US)

(72) Inventor: Daniel C. SANTANA, Sacramento, CA (US)

(21) Appl. No.: 19/675,166

(22) Filed: May 12, 2026

Related U.S. Application Data
(60) Provisional application No. 63/805,140, filed on May 13, 2025.

(51) Int. Cl. A61B 8/00

(52) U.S. Cl. CPC A61B 8/40; A61B 8/461

(57) ABSTRACT

Various embodiments of a tendon imaging apparatus and method are described herein. The apparatus may include a base configured for supporting at least a portion of a patient's hand during imaging where a distal end of the base is designed to position one or more digits of the patient's hand at a predetermined angle. The apparatus may further include a movable portion configured to slide. The movable portion may be coupled with the base and repositionable between at least a first position and a second position. The apparatus may further include a restraint coupled with the movable portion and configured to engage a digit of the patient's hand such that a force is applied to the digit. The force may be based at least in part on a configuration of the apparatus.

### CROSS-REFERENCES TO RELATED APPLICATIONS

This application claims the benefit of U.S. Provisional Patent Application No. 63/805,140, filed May 13, 2025, the entire contents of which are hereby incorporated by reference for all purposes in its entirety.

### BACKGROUND OF THE INVENTION

Various methods of imaging often require particularized positioning and/or immobilization of a patient or a portion of the patient's body. In particular, established approaches for imaging a patient's hand often require instructing the patient to maintain a set position during the procedure. However, such approaches can be challenging to reproduce and may be limited by the mobility of the patient.

### BRIEF SUMMARY OF THE INVENTION

Systems and methods of the present disclosure are related to a device for imaging a patient's hand and particularly, a tendon imaging device for facilitating the positioning of a digit of a patient's hand to allow sufficient flexion of the flexor tendon for imaging.

When conducting an imaging study of the flexor tendon system of a patient's hand, conventional practices may involve instructing a patient to maintain a specific hand position. For example, when conducting an imaging study of the flexor tendon system, standard procedure often includes instructing a patient to position their hand in a particular arrangement defined by a goniometer and to hold said position during imaging. However, such approaches often require repeated measurements and ongoing instructions to the patient to adjust hand or digit position, which can affect the repeatability and validity of the procedure. Embodiments of the present disclosure provide a tendon imaging apparatus for conducting imaging studies of the hand that utilizes a particularized structure to orient and restrain a digit of a patient, thereby imposing particularized positioning to sufficiently extend the tendons in the hand and maintain said position for imaging. As a result, the apparatus creates standardized positioning without the need for measurements and instruction to the patient to alter the positioning and, in turn, improves the reproducibility and validity of the imaging.

### BRIEF DESCRIPTION OF THE DRAWINGS

FIG. 1 illustrates a perspective view of a tendon imaging apparatus supporting a patient's hand, according to various embodiments;

FIG. 2 illustrates another perspective view of the tendon imaging apparatus, according to various embodiments;

FIG. 3 illustrates a side view of the tendon imaging apparatus, according to various embodiments;

FIG. 4A illustrates a top view of the tendon imaging apparatus in a first configuration, according to various embodiments;

FIG. 4B illustrates the top view of the tendon imaging apparatus in a second configuration, according to various embodiments;

FIG. 5A illustrates imaging the flexor tendon in a hand without implementing the tendon imaging apparatus, according to various embodiments;

FIG. 5B illustrates imaging the flexor tendon in a hand while implementing the tendon imaging apparatus, according to various embodiments; and

FIG. 6 illustrates a method for utilizing the tendon imaging apparatus for imaging, according to various embodiments.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION OF THE INVENTION

The figures and the following description relate to various embodiments by way of illustration only. It should be noted that from the following discussion, alternative embodiments of the structures and methods disclosed herein will be readily recognized as viable alternatives that may be employed without departing from the principles of the claimed invention.

Reference will now be made in detail to several embodiments, examples of which are illustrated in the accompanying figures. It is noted that wherever practicable similar or like reference numbers may be used in the figures and may indicate similar or like functionality. The figures depict embodiments of the present invention for purposes of illustration only. One skilled in the art will readily recognize from the following description that alternative embodiments of the structures and methods illustrated herein may be employed without departing from the principles of the invention described herein.

Systems and methods of the present disclosure provide a tendon imaging apparatus for positioning digit(s) during an imaging study of the flexor tendon system of a patient's hand. Conventional methods of imaging the flexor tendon system often require instructing the patient to maintain their hand in a set position. However, such approaches are difficult to consistently repeat, are limited by the mobility of the patient, and thus negatively impact the validity of the imaging. Embodiments of the present disclosure provide a base designed to support at least a portion of a patient's hand and configured to position one or more digits at a suitable angle. A restraint may be coupled with an adjustable member and designed to engage a digit of the patient's hand such that application of a counter-resistance to the digit by the restraint may allow the digit to be positioned at a suitable angle for imaging. By controlling the position and the amount of force applied to the digit, the device can help ensure that the tendons are presented in a way that is both consistent and repeatable from one imaging session to another. This reduces the need for repeated instructions or manual adjustments by the clinician, making the process smoother for both the patient and the healthcare provider.

Although the tendon imaging apparatus is described herein as being implemented to improve the quality and repeatability of an imaging study of the flexor tendon system in the hand, the tendon imaging apparatus may be used in other medical procedures as well. Any situation where precise and stable positioning of the hand, fingers, or other portion of a patient's body is desirable could potentially benefit from the disclosed apparatus. By standardizing how a portion of a patient's body is positioned, the device can help make results more reliable, easier to compare over time, and less dependent on the cooperation or physical capabilities of individual patients.

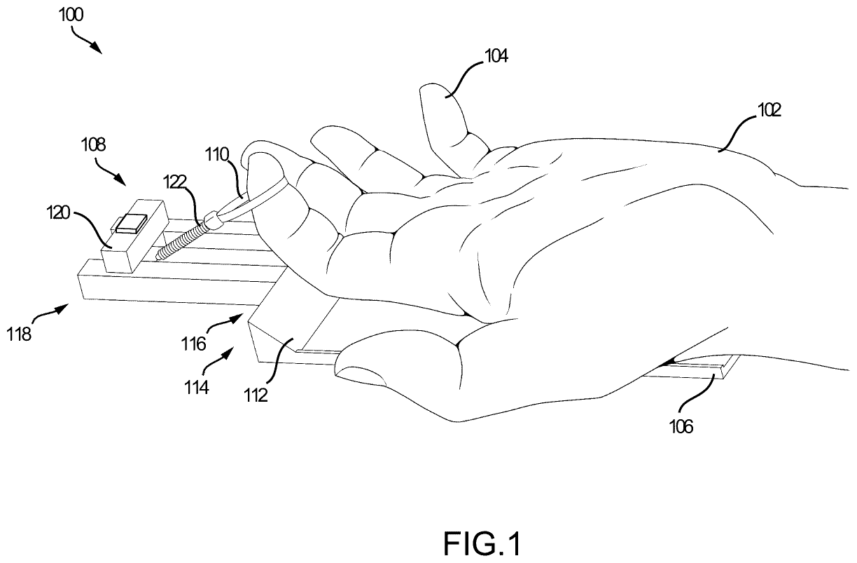

FIG. 1 illustrates a perspective view of a tendon imaging apparatus 100 supporting a patient's hand 102, according to various embodiments of the present disclosure. The tendon imaging apparatus 100 may include more or less components than those shown here and may include any combination of components described herein. Components having the same numbering shown in other figures are assumed to have the same configuration as the components described in relation to this figure.

The tendon imaging apparatus 100 may be configured to promote the stable and repeatable positioning of a patient's hand 102 and/or one or more digits 104 during imaging procedures. For example, the tendon imaging apparatus 100 may be utilized to maintain the position of a restrained digit 104 such that the metacarpophalangeal (MCP) joint is in 0-degree extension, the proximal interphalangeal (PIP) joint is in 40-degree flexion, and/or the distal interphalangeal (DIP) joint is in 10-degree flexion. By facilitating this alignment, the tendon imaging apparatus 100 may provide for greater separation between the flexor tendons and the bones in the hand during imaging, potentially improving the clarity of the image and allowing easier assessment of the flexor tendon system. One or more components of the tendon imaging apparatus 100 may be made from a variety of materials including but not limited to aluminum, stainless steel, polycarbonate, polypropylene, or other metallic materials, composite materials, or other similar materials. In various embodiments, at least a portion of the tendon imaging apparatus 100 is made from a lightweight material such as aluminum. In various embodiments, the tendon imaging apparatus 100 may be constructed with antimicrobial coatings or materials to enhance infection control in clinical settings.

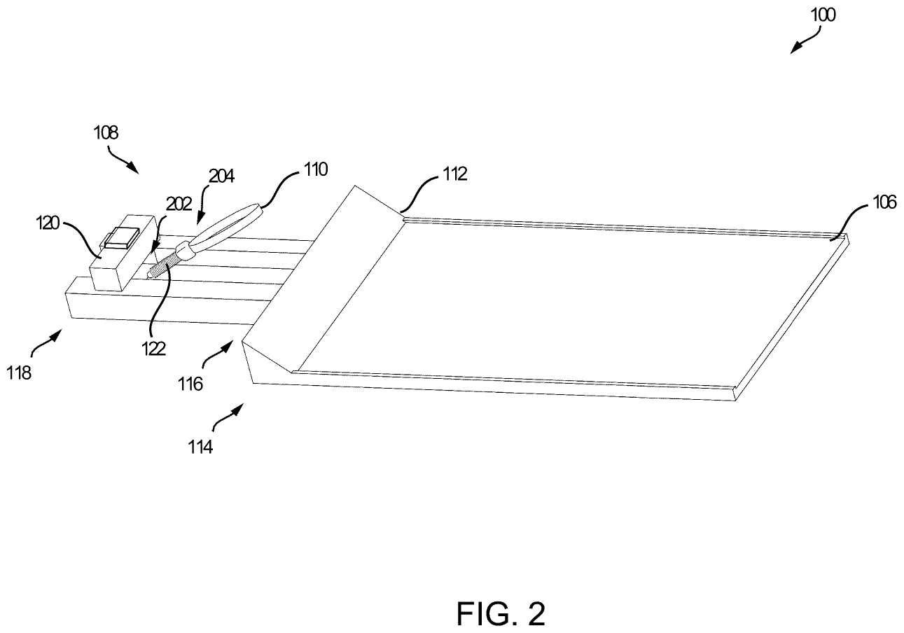

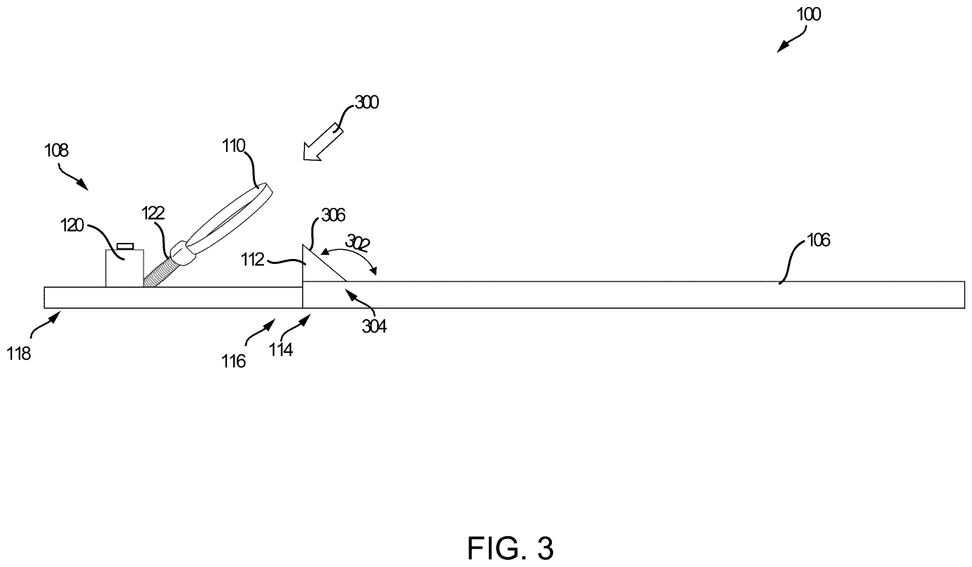

FIG. 2 illustrates another perspective view of the tendon imaging apparatus 100, according to various embodiments of the present disclosure. The tendon imaging apparatus 100 may include a base 106, an adjustable member 108, and a restraint 110. In various embodiments, the base 106 may be a base plate coupled with at least the adjustable member 108 and the restraint 110. In various embodiments, the base 106 may be integrated into a fixture such as a table or other surface on which the patient's hand 102 is positioned. In an example, the base 106 is a part of a container that can be filled with water where the restraint 110 is coupled with a wall of the container. The base 106 may be contoured or otherwise shaped to support at least a portion of a patient's hand 102 during imaging. For example, the base 106 may be designed to support the dorsal surface of a hand 102 such that a patient may rest their hand 102 on the base 106 during imaging. As discussed in more detail with respect to FIG. 3, the base 106 may include an angled portion 112. The angled portion 112 may be provided as a sloped or ramped feature, and may be designed to facilitate the precise positioning of one or more digits 104 of a hand 102 for imaging. In various embodiments, the angled portion 112 may be located at or near a distal end 114 of the base 106. For example, the angled portion 112 may be provided as a ramp structure located at the distal end 114 of the base 106. In various embodiments, the base 106 may incorporate adjustable or removable supports that conform to different hand shapes or sizes, such as contoured inserts, inflatable bladders, or gel pads. The base 106 may also include channels or grooves to facilitate alignment of individual digits 104 or accommodate wires from physiological sensors.

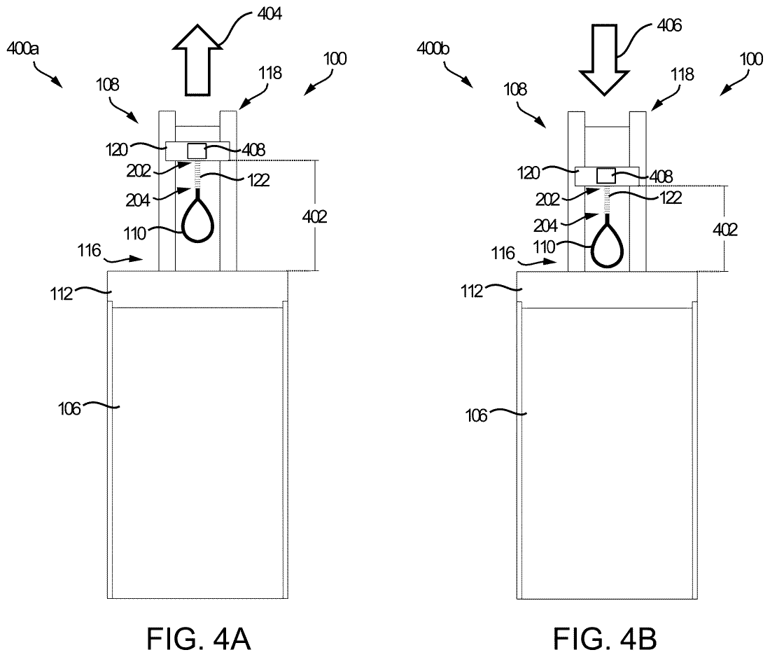

The tendon imaging apparatus 100 may further include an adjustable member 108 with a first end 116 and a second end 118. The adjustable member 108 may be positioned to extend beyond an end of the base 106. For example, as illustrated in FIG. 2, the adjustable member 108 may extend past the distal end 114 of the base 106. The adjustable member 108 may further include a movable portion 120. The movable portion 120 may be coupled with the adjustable member 108 and may be configured to slide with respect to the adjustable member 108. For example, the movable portion 120 may be coupled with the adjustable member 108, such that the movable portion 120 can slide towards and away from the distal end 114 of the base 106 along the length of the adjustable member 108. This sliding feature can allow for adjustment of the position of the movable portion 120 between at least the first end 116 and the second end 118 of the adjustable member 108. For example, the movable portion 120 may be moved to any suitable position between the first end 116 and the second end 118, accommodating variations in hand and/or finger size among different patients. In various embodiments, the movable portion 120 may be secured at a chosen position along the adjustable member 108. Thus, when a digit 104 is engaged with the restraint 110, the movable portion 120 may be held in position with sufficient fastening force so that the movable portion 120 does not change positions during assessment of the hand 102. As discussed in more detail with respect to FIG. 4A, the movable portion 120 may include a fastener 408 to secure the movable portion 120 to the adjustable member 108. For example, the movable portion 120 may include a screw or clamp that can be tightened to hold the movable portion 120 in place at a chosen location (e.g., distance 402) during assessment. As a result, the apparatus 100 may be adapted to different anatomical needs while providing stability for the digit 104 during imaging.

The tendon imaging apparatus 100 may further include a restraint 110. The restraint 110 may be designed to engage and secure a digit 104 of a patient's hand 102. In various embodiments, the restraint 110 may be a loop-type restraint, such as a zip-tie, as illustrated in the Figures. However, while the restraint 110 is discussed and illustrated herein as a loop-type restraint, such as a zip-tie, any suitable restraint 110 may be used to engage a digit 104. For example, a strap or cuff may be designed to engage a digit 104. The restraint 110 may be a non-elastic element. In various embodiments, the restraint 110 may be coupled with the movable portion 120 of the adjustable member 108 so that the position of the restraint 110 can be adjusted along the adjustable member 108 to suit the length of the digit 104 being engaged. For example, one end of the restraint 110 may be attached to the movable portion 120, thus when the movable portion 120 is slid along the adjustable member 108, the restraint 110 may be positioned closer or farther away from the distal end 114 of the base 106 based on the particular digit length of the patient. Once in place, the movable portion 120 may be locked in place, ensuring that a digit 104 secured via the restraint 110 may remain in a desired orientation through an imaging procedure. The restraint 110 may be utilized to impart a resistive force (shown in FIG. 3 as force 300), to a digit 104 engaged by the restraint 110. For example, when a digit 104 is being held by the restraint 110, a force 300 is being applied to the digit 104 such that the digit 104 is flexed by a predetermined amount to extend and maintain the hand 102 in a standardize posture for imaging. In various embodiments, the restraint 110 may include padding, such as memory foam or silicone, to reduce pressure points and enhance patient comfort. In various embodiments, the restraint 110 may be detachable and sterilizable, or provided in single-use disposable form to address infection control requirements.

In various embodiments, the restraint 110 may be coupled with the movable portion 120 of the adjustable member 108 via a force-applying element (e.g., spring 122). For example, as illustrated in FIG. 2, the first end 202 of the spring 122 may be coupled with the movable portion 120 and the second end 204 of the spring 122 may be coupled with the restraint 110. Although the force-applying element is discussed and illustrated herein as a spring 122, it should be understood that the force-applying element may comprise any suitable mechanism capable of imparting force, such as tension, compression, or other mechanical force, to a patient's hand 102 and/or digit 104 when the apparatus 100 is in use, including but not limited to elastic bands, pneumatic or hydraulic actuators, weights, or motorized devices. In various embodiments, the force-applying element (e.g., spring 122) may be a tension spring. Thus, when a digit 104 is engaged with the restraint 110, the force-applying element (e.g., spring 122) may further impart a resistive force 300 to the digit 104, where the resistive force 300 is based at least in part on a force characteristic (e.g., the tension) of the force-applying element (e.g., spring 122). In various embodiments, the force-applying element (e.g., spring 122) may be an extension spring. The extension spring (e.g., spring 122) may then be coupled with the tendon imaging apparatus 100 such that the force 300) is imparted to a digit 104 by pulling on the extension spring (e.g., spring 122) with the digit 104. For example, as illustrated in FIG. 1, a digit 104 positioned in the tendon imaging apparatus 100 may extend the spring 122 by applying a force 300 to the spring 122 (e.g., the length of the spring 122 may increase). In comparison, when a digit 104 is not engaged with the restraint 110, the length of the spring 122 may be shorter, as illustrated in FIGS. 2 and 3. In various embodiments, the extension spring (e.g., spring 122) may be coupled with the tendon imaging apparatus 100 and positioned proximate to the palmar surface of the hand 102. Thus, to extend the spring 122, the digit 104 may extend away from the palmar surface. In various embodiments, the force-applying element (e.g., spring 122) may be selected based on the force a patient can pull with their digit 104. For example, the maximum force a patient can pull with their digit 104 may be determined. Then, a spring 122 with a desired tension may be selected based on the maximum force, such that the force 300 imparted by the spring 122 to the digit 104 is sufficient to achieve a desired tension in the digit 104 (e.g., a chosen tension in the flexor tendons to be imaged). Although the restraint 110 is described and illustrated herein as being coupled with a spring 122, the restraint 110 may be used alone or coupled with any suitable component capable of imparting a resistive force 300 to a digit 104 when engaged with the restraint 110. In various embodiments, the restraint 110 may be coupled with the movable portion 120 via the spring 122 and a strain gauge where the strain gauge is configured to provide a calibrated force output to be applied to a digit 104 engaged by the restraint 110. In various embodiments, the apparatus 100 may include interchangeable spring cartridges with different stiffness levels to further customize resistance for specific patient populations or diagnostic needs.

FIG. 3 illustrates a side view of the tendon imaging apparatus 100, according to various embodiments of the present disclosure. The apparatus 100 may be designed to facilitate anatomically standardized and reproducible positioning of a patient's hand 102 and/or one or more digits 104 during imaging procedures. The apparatus 100 may be particularly suited for diagnostic imaging techniques, including ultrasound, where consistent joint positioning can enhance image quality and clinical assessment.

The base 106 of the apparatus 100 may include an angled portion 112 extending from a location 304 at or near the distal end 114 of the base 106. The angled portion 112 may be shaped or contoured to facilitate the stable placement of one or more digits 104 for imaging. In various embodiments, the angled portion 112 may be configured such that the dorsal side of a PIP joint of a digit 104 may rest at a location 304 where the angled portion 112 meets the base 106. As a result, one or more digits 104 may be oriented in a standardized and reproducible manner for imaging. For example, the angled portion 112 may extend from the base 106 such that the angled portion 112 is suitable to position a digit 104 where the MCP joint of the digit 104 is in 0-degree extension, the PIP joint of the digit 104 (e.g., positioned at the location 304 where the angled portion 112 meets the base 106) is in 40 degrees of flexion, and the DIP joint of the digit 104 is in 10 degrees of flexion. Such precision in joint alignment can allow for a more consistent separation between the flexor tendons and the bones during imaging, which may lead to improved visualization and assessment of the tendon system.

The sizing and geometry of the angled portion 112 may be selected based on various anatomical considerations, such as the dimensions of a patient's hand 102 and/or digit(s) 104 supported by the base 106. In various embodiments, the length of the hypotenuse 306 of the angled portion 112 may be based at least in part on the length of the middle phalanx (e.g., digit 104) of a hand 102 to be imaged. In particular, the angled portion 112 may be designed such that the length of the hypotenuse 306 is less than the length of the middle phalanx (e.g., digit 104) of a hand 102 to be imaged. This configuration can help ensure that the desired degree of flexion is obtained when the digit 104 is positioned for imaging. In various embodiments, the angle at which the angled portion 112 extends from the base 106 (e.g., a predetermined angle 302) may be chosen to provide the appropriate joint alignment for imaging. For example, the angled portion 112 may extend at a 45-degree angle with respect to the base 106. In various embodiments, a suitable angle 302 at which the angled portion 112 extends from the base 106 may be between 35 degrees and 45 degrees. In various embodiments, a suitable range for the angle 302 at which the angled portion 112 extends from the base 106 may be between 35 degrees to 45 degrees. In various embodiments, a suitable range for the angle 302 may be between 15 degrees to 70 degrees. By positioning digit(s) 104 along the angled portion 112 at a predetermined angle 302, the tendon imaging apparatus 100 can standardize the joint angle position of the digit 104, which can improve the reproducibility and the validity of imaging. This is an improvement on conventional methods of imaging the hand 102 where a patient is instructed to maintain a set position for their hand 102 and/or digits 104 and no positioning is imposed.

The tendon imaging apparatus 100 may be configured to apply a counter-resistance (e.g., force 300) to an engaged digit 104. Specifically, the restraint 110 may be utilized to engage a digit 104 of a hand positioned on the tendon imaging apparatus 100 and, in turn, the restraint 110 may impart a resistive force 300 to the engaged digit 104. The force 300 may be utilized to position digit(s) 104 and/or the hand 102 such that the digit(s) 104 and/or the hand 102 remain in a chosen position for imaging. The force 300 may be determined based on the force 300 suitable to maintain the digit(s) 104 in a chosen orientation for imaging. For example, a force 300 may be chosen to position a restrained digit 104 such that the MCP joint is in 0-degree extension, the PIP joint is in 40-degree flexion, and/or the DIP joint is in 10-degree flexion. The application of a counter-resistive force 300 may be particularly beneficial when assessing for conditions such bowstringing in the flexor tendon system of a hand 102. Bowstringing may occur when the tendons, which are normally held close to the bone by a pulley system, pull away from the bone. When imaging bowstringing of a tendon in a patient's hand 102, the tendon is accentuated by putting the tendon under tension (e.g., such as by applying a force 300 to a digit 104 of the hand 102), which can be done by flexing the digit 104 associated with the tendon to be imaged. However, a patient, especially one suffering from bowstringing of the flexor tendon, may only be capable of flexing their hand 102 and digit(s) 104 a limited degree, and it may be difficult to hold such a position for a prolonged period of time and/or to reproduce the position for repeated imaging. By further applying a force 300 to a digit 104 via the tendon imaging apparatus 100 and flexing the PIP joint, bowstring of the flexor tendon of the digit 104 may be accentuated, allowing for clearer imaging that is easily repeatable without depending on the capabilities of the patient.

In various embodiments, the tendon imaging apparatus 100 may be designed with a low profile. Specifically, the tendon imaging apparatus 100 may be designed such that when the dorsal surface of a hand 102 is supported on the base 106, the palmar surface of the hand 102 remains relatively unobstructed. Thus, imaging components, such as an ultrasound transducer, may be easily utilized during imaging of the tendons without being blocked. For example, when a digit is engaged in the restraint 110 of the tendon imaging apparatus 100, a physician can easily use an ultrasound transducer on the palmar surface of the hand 102 without being obstructed by the digit(s) 104 or portions of the tendon imaging apparatus 100. By minimizing obstructions, the apparatus 100 may support high-quality imaging and clinical flexibility during diagnostic procedures.

FIG. 4A illustrates a top view of the tendon imaging apparatus 100 in a first configuration 400a, and FIG. 4B illustrates the top view of the tendon imaging apparatus 100 in a second configuration 400b, according to various embodiments of the present disclosure. The apparatus 100 may include a movable portion 120 that is operably coupled with the adjustable member 108. The movable portion 120 may be configured to slide with respect to the adjustable member 108, such that the movable portion 120 is repositionable between the first end 116 and the second end 118 of the adjustable member 108. Specifically, the distance 402 of the movable portion 120 from the distal end 114 of the base 106 can be reconfigured. This adjustability may permit the apparatus 100 to accommodate hands and digits 104 of varying sizes, as well as to tailor the mechanical loading or positioning for specific imaging procedures. As illustrated in FIG. 4A, the movable portion 120 may slide in a first direction 404 away from the base 106 to increase the distance 402 between the movable portion 120 and the distal end 114 of the base. Moreover, as illustrated in FIG. 4B, the movable portion 120 may slide in a second direction 406 towards the base 106 to decrease the distance 402. This design can provide a range of possible positions for the movable portion 120, allowing for individualized adjustments for each patient.

In various embodiments, to secure the movable portion 120 at a desired location (e.g., distance 402) along the adjustable member 108, the apparatus 100 may include a fastener 408. For example, the movable portion 120 may include a screw, clamp, or similar mechanism that can be tightened to hold the movable portion 120 in place at a desired location (e.g., distance 402). The fastener 408 may be configured to provide suitable fastening force to hold the movable portion 120 in place when a digit 104 is engaged by the restraint 110. For example, when the restraint 110 is imparting a resistive force 300 to an engaged digit 104, the fastener 408 may secure the movable portion 120 at a location (e.g., distance 402) on the adjustable member 108 with suitable fastening force to prevent the force 300 between the restraint 110 and the digit 104 from causing the movable portion 120 to move along the adjustable member 108. This may help ensure that the selected orientation of the hand and/or digits 104 remain stable throughout the imaging procedure and contributes to the reproducibility and reliability of the apparatus 100.

In various embodiments, the force 300 applied to the digit 104 when engaged with the restraint 110 may be based at least in part on the distance 402 of the movable portion 120 from the distal end 114 of the base 106. For example, to increase the force 300 applied to a digit 104 engaged with the restraint 110, the movable portion 120 may slide in the first direction 404 away from the base 106. To decrease the force 300 applied to a digit 104 engaged with the restraint 110, the movable portion 120 may slide in the second direction 406 towards the base 106. As a result, the force 300 applied to a digit 104 may be adjusted to achieve the desired position of the digit(s) 104 for imaging. Additionally or alternatively, the force 300 applied to the digit 104 when engaged with the restraint 110 may be based at least in part on a force characteristic of the force-applying element (e.g., tension of the spring 122) coupled with the restraint 110. The force-applying element (e.g., spring 122), with the first end 202 coupled with the movable portion 120 and the second end 204 coupled with the restraint 110, may impart a force 300 to the digit 104 based at least in part on at least one force characteristic, such as tension, of the force-applying element (e.g., spring 122). In various embodiments, the force-applying element (e.g., spring 122) may be selected to provide a range of counter-resistive force 300. For example, a first spring 122 may be selected to provide a first lower counter-resistive force 300 to a digit 104 of a pediatric patient and a second spring 122 may be selected to provide a second stiffer counter-resistive force 300 to a digit 104 of an adult patient. This modularity may help ensure that the apparatus 100 remains versatile across a broad range of clinical applications and patient populations.

In various embodiments, the tendon imaging apparatus 100 may include one or more sensors. The sensors may be used to calculate one or more force characteristics (e.g., the force 300 exerted by) of the digit 104 engaged by the restraint 110. For example, a thin film pressure sensor or a strain gauge may be integrated with the restraint 110 or spring 122 assembly. In turn, the sensors may be configured to provide real-time feedback about the magnitude of the force characteristic (e.g., force 300), such as by measuring the pressure applied by the digit 104 or by detecting the deformation of the spring 122. In various embodiments, a real time display may be included to provide force characteristic readings related to the one or more force characteristics (e.g., force 300) exerted by the engaged digit 104. For example, as previously discussed, the restraint 110 may be coupled with the movable portion 120 via a spring 122 and a strain gauge where the strain gauge is configured to provide a calibrated force output to be applied to a digit 104 engaged by the restraint 110. Said strain gauge may be implemented with force recording and a real time display to provide further clarity to the force 300 being applied to the digit 104 engaged by the restraint 110. As a result, clinicians may be able to monitor and adjust one or more force characteristics (e.g., the force 300) as needed during imaging procedures. In various embodiments, the sensors may include accelerometers or gyroscopes to detect movement or vibration during imaging, allowing for automated alerts if the patient's hand shifts out of position.

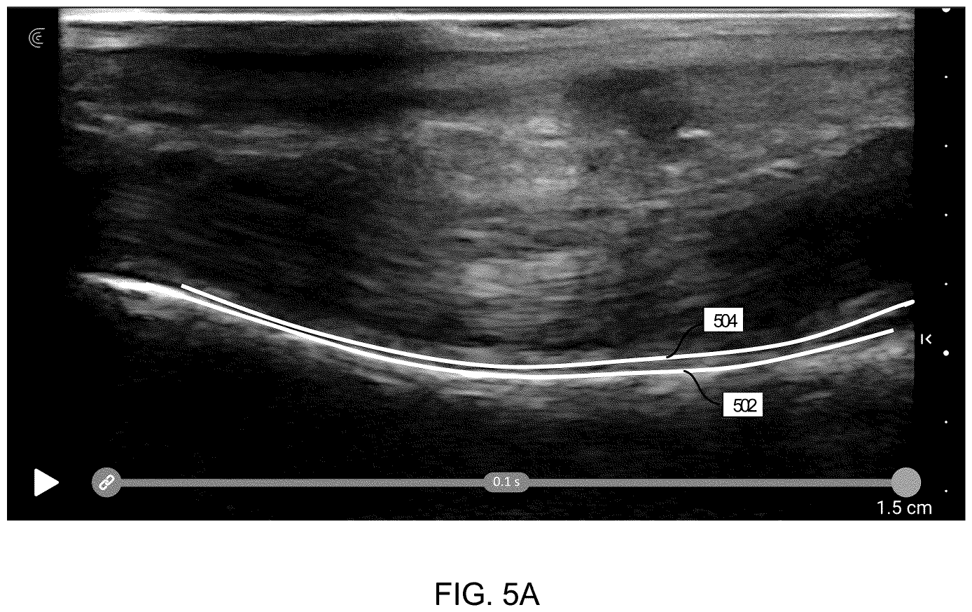

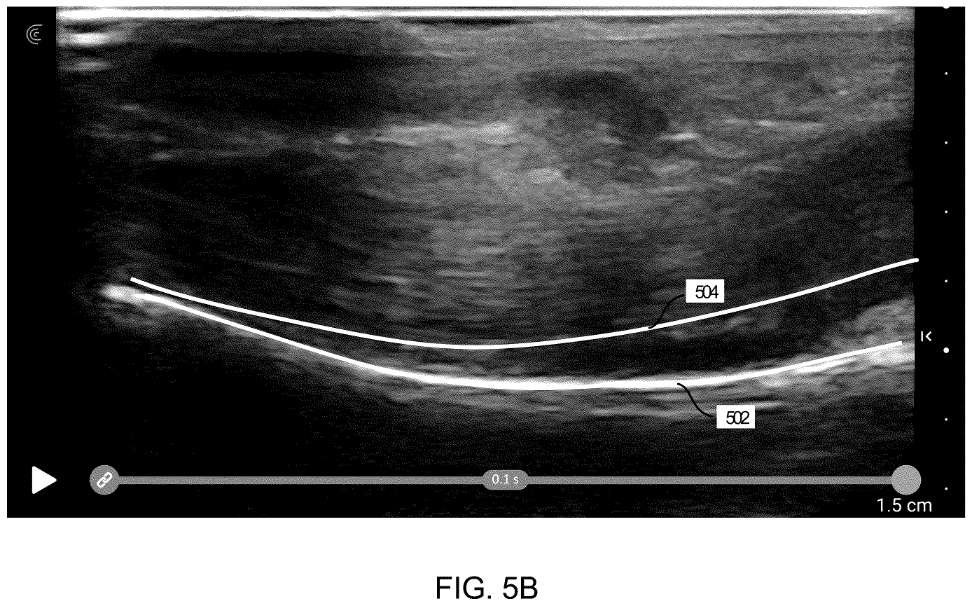

FIG. 5A illustrates imaging the flexor tendon in a hand 102 without implementing the tendon imaging apparatus 100, and FIG. 5B illustrates imaging the flexor tendon in a hand 102 while implementing the tendon imaging apparatus 100, according to various embodiments of the present disclosure. FIGS. 5A and 5B demonstrates the anatomical relationship between the volar cortex bone (line 502) of a hand 102 and the dorsal border of the flexor tendons (line 504) as visualized under different imaging conditions. Due to the pulley system in a hand 102, as a digit 104 bends, the flexor tendon 504 stays relatively close to the bone 502 and pulls away a small amount from the bone 502 the closer the tendon 504 is to the digit 104 (towards the right of the illustration in FIGS. 5A and 5B). Under typical circumstances, this separation is minimal due to the integrity of the pulley system. If the pulley system is compromised or injured, the tendon 504 may separate away from the bone 502 by a larger degree than normal when there is tension on the tendon 504, a phenomenon commonly referred to as bowstringing. Thus, one way to diagnose an injury to the pulley system of the hand 102 is to assess for bowstringing by measuring the distance between the tendon 504 and the bone 502 when under tension.

FIG. 5A illustrates a hand 102 where the flexor tendon 504 is under active tension from the patient manually bending the digit 104. As the flexor tendon 504 moves towards the digit 104 (e.g., towards the right of the image), the tendon 504 starts to curve away from the bone because the PIP joint is flexed. However, the separation between the tendon 504 and the bone 502 is minimal and limited by the flex of the digit 104 making the tendon 504 and bone 502 separation difficult to image. This minimal separation can make it difficult to produce clear and consistent images of the distance between the tendon 504 and the bone 502. Moreover, since the flexion of the digit 104 is not standardized (e.g., dependent on providing instructions to a patient to maintain a set position defined by a goniometer), the distance between the tendon 504 and the bone 502 can be different values between imaging sessions based on the varied flexion of the digit 104. As a result, repeatability and reliability of imaging the hand 102 in this manner is negatively impacted making longitudinal assessment or cross-patient comparison more challenging.

FIG. 5B, in contrast, illustrates imaging of the hand 102 when the tendon imaging apparatus 100 is engaged. In this configuration, a controlled and standardized force 300 is applied to the digit 104, resulting in a greater separation between the flexor tendon 504 and the bone 502 compared to manual flexion alone (as illustrated in FIG. 5A). This increased distance may yield a clearer, more discernable image of the tendon 504 and bone 502 that can facilitate improved evaluation of the pulley system of the hand 102. Moreover, since the tendon imaging apparatus 100 implements standardized positioning of the hand 102 and digit(s) 104, the image illustrated in FIG. 5B can be easily reproduced showing a similar distance between the bone 502 and tendon 504 between distinct imaging sessions.

The tendon imaging apparatus 100 may be formed from a selection of materials to optimize, function, durability, and/or patient comfort. As previously discussed, one or more components of the tendon imaging apparatus 100 may be made from materials including but not limited to aluminum, stainless steel, polycarbonate, polypropylene, or other metallic materials, composite materials, or other similar materials. For example, the base 106 may be made from a plastic or metal material. In various embodiments, the base 106 may be made from a plastic material making the base 106 lightweight and easier to clean. In various embodiments, the adjustable member 108 may at least partially be made from a metal material. Specifically, the adjustable member 108 may include one or more metal rails to provide rigidity and support as well as allow the movable portion 120 to easily slide along the one or more metal rail of the adjustable member 108. In various embodiments the restraint 110 may be made of textile. For example, the restraint 110 may be a textile loop or strap. In various embodiments, the force-applying element (e.g., spring 122) may be replaced or supplemented with an electronic strain gauge configured to provide a calibrated force output.

In various embodiments, the tendon imaging apparatus 100 may be designed for use in a sterile field, with all patient-contacting components autoclavable or compatible with chemical sterilant. Alternatively or additionally, the tendon imaging apparatus 100 may be manufactured in single-use, disposable form settings. In various embodiments, the tendon imaging apparatus 100 may be adapted for pediatric use, with smaller dimensions and reduced resistance levels.

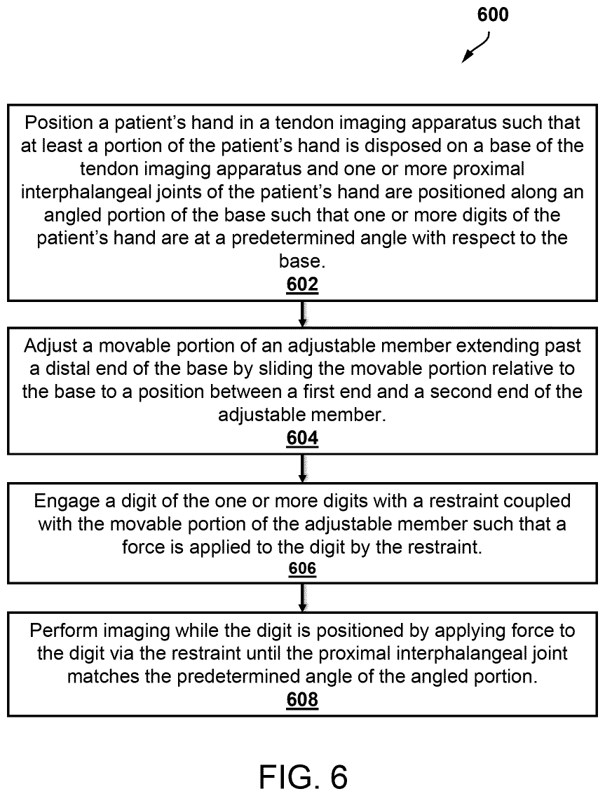

FIG. 6 illustrates a method 600 for utilizing the tendon imaging apparatus 100 for imaging, according to various embodiments of the present disclosure. The method 600 may provide a systematic approach to preparing and positioning a patient's hand 102 for diagnostic imaging, with the goal of standardizing flexor tendon tension and improving the reliability of clinical assessments.

The method may include operation 602, in which a patient's hand 102 is positioned in the tendon imaging apparatus 100. The hand 102 may be positioned such that at least a portion of the hand 102 is disposed on a base 106. In various embodiments, one or more PIP joints of the hand 102 may be located at a location 304 along the angled portion 112 of the base 106. For example, the dorsal surface of the hand 102 may rest on the base 106 during imaging and one or more PIP joints of the hand 102 may rest at a location 304 proximate to where the angled portion 112 meets the base 106. In turn, the one or more digits 104 of the hand 102 can be positioned at a predetermined angle 302 (imposed by the angled portion 112) with respect to the base 106. For example, the angled portion 112 may have an angle 302 of 40 degrees such that, when the patient's hand 102 is positioned in the tendon imaging apparatus 100, at least one digit 104 may be positioned along the angled portion 112 where the PIP joint is at an angle 302 of 40 degrees.

The method 600 may further include operation 604, in which the movable portion 120 of the adjustable member 108 may be adjusted. The adjustable member 108 may be coupled with the base 106 such that the adjustable member 108 extends past the distal end 114 of the base 106. To adjust the tendon imaging apparatus 100, the movable portion 120 may slide along the adjustable member 108 relative to the base 106 to a position between a first end 116 and a second end 118 of the adjustable member 108. For example, the movable portion 120 may be adjusted by sliding the movable portion 120 in a first direction 404 away from the base 106 to increase the distance 402 between the movable portion 120 and the base 106. Moreover, the movable portion 120 may be adjusted by sliding the movable portion 120 in a second direction 406 towards the base 106 to decrease the distance 402 between the movable portion 120 and the base 106. This adjustment may permit fine-tuning of the apparatus 100 to accommodate different hand sizes, finger lengths, or desired levels of tendon tension.

The method 600 may further include operation 606, in which a digit 104 of the one or more digits 104 is engaged with a restraint 110. For example, a restraint 110, such as a loop-type restraint, may be wrapped around the tip of a digit 104 to restrain the digit 104. The restraint 110 may be coupled with the movable portion 120 of the adjustable member 108 and may apply a force 300 to the engaged digit 104. The force 300 may be utilized to position and hold the one or more digits 104 in a standardized position for imaging. As a result, tension may be set in the tendons of the hand 102 resulting in a larger separation between the bone 502 and the flexor tendons 504 allowing for clear imaging of the tendons 504 and the pulleys that support said tendons 504. In various embodiments, the force 300 may be based at least in part on the distance 402 between the movable portion 120 and the distal end 114 of the base 106. For example, to increase the force 300 applied to an engaged digit 104, the movable portion 120 may be slid in the first direction 404 away from the base 106. To decrease the force 300 applied to an engaged digit 104, the movable portion 120 may be slid in the second direction 406 towards the base 106. Thus, the force 300 applied to a digit 104 may be adjusted by sliding the movable portion 120 along the adjustable member 108 to achieve the desired position of the digit(s) 104 for imaging. Additionally or alternatively, a force-applying element (e.g., spring 122) may be included such that a first end 202 of the force-applying element (e.g., spring 122) is coupled with the movable portion 120 and a second end 204 of the force-applying element (e.g., spring 122) is coupled with the restraint 110. In turn, the force 300 applied to the engaged digit 104 may be based at least in part on one or more force characteristics (e.g., tension) of the force-applying element (e.g., spring 122). In various embodiments, the force-applying element (e.g., spring 122) may be calibrated based on the maximum force a patient can pull and/or push with their digit 104. For example, the maximum force a patient can pull or push with their digit 104 may be determined. Then, the tension of the spring 122 may be calibrated based on the maximum force so that the force 300 imparted by the spring 122 to the digit 104 is sufficient to achieve a desired tension in the digit 104 (e.g., a chosen tension in the flexor tendons to be imaged). In various embodiments, the force-applying element (e.g., spring 122) may be calibrated to provide a range of counter-resistive force 300. For example, the spring 122 may be calibrated to provide a first counter-resistive force 300 to the digit 104 to position the digit(s) 104 as desired. However, if the resulting position of the digit(s) 104 is not as desired, the spring 122 may be recalibrated to provide a second counter-resistive force 300 to the digit 104.

The method 600 may include operation 608, in which imaging is performed while the digit 104 is positioned by applying force 300 to the digit 104 via the restraint 110 until the proximal interphalangeal joint matches the predetermined angle 302 of the angled portion 112. For example, a patient's digit 104 may be flexed by a predetermined amount, which corresponds to the predetermined angle 302, to extend the flexor tendon corresponding to the digit 104 for imaging. As discussed, when imaging the bowstringing of a tendon in a patient's hand 102, the tendon may be accentuated by flexing digit(s) 104 of the hand 102. However, the degree of flexing of digit(s) 104 is limited by the mobility of the patient. Thus, by implementing the tendon imaging apparatus 100 and putting the tendon under tension (e.g., such as by applying a force 300 to an engaged digit 104 of the hand 102), the tendon may be accentuated for imaging in a measurable, standardized, and repeatable manner.

In various embodiments, the tendon imaging apparatus 100 may be equipped with additional features to further enhance the method 600. For example, measurement markings or sensors may be included to record the precise position of the movable portion 120 and the magnitude of the force 300 applied. A strain gauge or similar sensor may be integrated with the restraint 110 or force-applying element (e.g., spring 122) to provide real-time feedback on force application, which may be displayed on a monitor for clinician reference. These optional features may support consistent documentation, reproducibility, and patient safety throughout the imaging process.

While particular embodiments and applications have been illustrated and described herein, it is to be understood that the embodiments are not limited to the precise construction and components disclosed herein and that various modifications, changes, and variations may be made in the arrangement, operation, and details of the methods and apparatuses of the embodiments without departing from the spirit and scope of the embodiments as defined in the appended claims.

Upon reading this disclosure, those of skill in the art will appreciate still additional alternative designs for the system. Thus, while particular embodiments and applications of the present invention have been illustrated and described, it is to be understood that the invention is not limited to the precise construction and components disclosed herein and that various modifications, changes and variations which will be apparent to those skilled in the art may be made in the arrangement, operation and details of the method and apparatus of the present invention disclosed herein without departing from the spirit and scope of the invention as defined in any claims drawn to the subject matter herein.

What is claimed is:

1. A tendon imaging apparatus for positioning a patient's digits during an imaging study, the apparatus comprising:
a base configured for supporting at least a portion of a patient's hand during imaging, the base including an angled portion extending from a distal end of the base at a predetermined angle with respect to the base and configured to position one or more digits of the patient's hand at the predetermined angle;
an adjustable member with a first end and a second end, the first end of the adjustable member coupled with the distal end of the base such that the adjustable member extends past the distal end of the base, the adjustable member including a movable portion repositionable between the first end and the second end of the adjustable member;
a force-applying element coupled with the movable portion of the adjustable member; and
a restraint coupled with the force-applying element and configured to engage a digit of the patient's hand such that a force is applied to the digit, wherein the force is based at least in part on a location of the movable portion along the adjustable member and a force characteristic of the force-applying element.

2. The tendon imaging apparatus of claim 1, wherein the predetermined angle is between 35 degrees and 45 degrees with respect to the base.

3. The tendon imaging apparatus of claim 1, further comprising:
one or more sensors configured to determine the force applied to the digit engaged by the restraint.

4. The tendon imaging apparatus of claim 1, wherein the restraint further includes a strain gauge configured to determine the force applied to the digit engaged by the restraint.

5. The tendon imaging apparatus of claim 1, wherein the restraint is a loop-type restraint.

6. A tendon imaging apparatus for positioning a patient's digits during an imaging study, the apparatus comprising:
a base configured for supporting at least a portion of a patient's hand during imaging, a distal end of the base designed to position one or more digits of the patient's hand at a predetermined angle;
a movable portion configured to slide, the movable portion coupled with the base and repositionable between at least a first position and a second position; and
a restraint coupled with the movable portion and configured to engage a digit of the patient's hand such that a force is applied to the digit, wherein the force is based at least in part on a configuration of the apparatus.

7. The tendon imaging apparatus of claim 6, wherein the joint is a proximal interphalangeal (PIP) joint of the digit engaged by the restraint.

8. The tendon imaging apparatus of claim 6, further comprising:
a spring coupled with the movable portion.

9. The tendon imaging apparatus of claim 8, wherein the force is based at least in part on a tension of the spring.

10. The tendon imaging apparatus of claim 6, wherein the base includes an angled portion extending from a distal end of the base at the predetermined angle with respect to the base.

11. The tendon imaging apparatus of claim 10, further comprising:
an adjustable member with a first end and a second end, the first end of the adjustable member coupled with the distal end of the base such that the adjustable member extends past the distal end of the base, the adjustable member including the movable portion.

12. The tendon imaging apparatus of claim 11, wherein at least the first position and the second position of the movable portion are located between the first end and the second end of the adjustable member.

13. The tendon imaging apparatus of claim 6, wherein the configuration of the apparatus includes a distance of the movable portion from a joint of the digit engaged by the restraint.

14. The tendon imaging apparatus of claim 6, further comprising:
one or more sensors configured to determine the force applied to the digit engaged by the restraint.

15. The tendon imaging apparatus of claim 6, wherein the restraint further includes a strain gauge configured to determine the force applied to the digit engaged by the restraint.

16. A method for positioning a patient's digits during an imaging study, the method comprising:
positioning a patient's hand in a tendon imaging apparatus such that at least a portion of the patient's hand is disposed on a base of the tendon imaging apparatus and one or more proximal interphalangeal joints of the patient's hand are positioned along an angled portion of the base such that one or more digits of the patient's hand are at a predetermined angle with respect to the base;
adjusting a movable portion of an adjustable member extending past a distal end of the base by sliding the movable portion relative to the base to a position between a first end and a second end of the adjustable member;
engaging a digit of the one or more digits with a restraint coupled with the movable portion of the adjustable member such that a force is applied to the digit by the restraint; and
performing imaging while the digit is positioned by applying force to the digit via the restraint until the proximal interphalangeal joint matches the predetermined angle of the angled portion.

17. The method of claim 16, further comprising: adjusting the force applied to the digit by repositioning the movable portion by sliding the movable portion along the adjustable member to vary tension in the flexor tendon.

18. The method of claim 17, wherein engaging the digit with the restraint comprises utilizing a loop-type restraint to secure the digit during imaging.

19. The method of claim 17, further comprising:
determining the force applied to the digit using a sensor coupled with the restraint; and
displaying the force via a real-time display.
